(19) United States
(12) Patent Application Publication
(10) Pub. No.: US 2004/0143147 A1
(43) Pub. Date: Jul. 22, 2004

(54) Process for producing alpha-olefins

(76) Inventors: Steven Dale Ittel, Wilmington, DE (US); David Allen Berg, Kingston (CA); John Charles Fisher, Kingston (CA)

Correspondence Address: E I DU PONT DE NEMOURS AND COMPANY, LEGAL PATENT RECORDS CENTER, BARLEY MILL PLAZA 25/1128, 4417 LANCASTER PIKE, WILMINGTON, DE, 19805, US

(21) Appl. No.: 10/702,685

(22) Filed: Nov. 6, 2003

Related U.S. Application Data
(60) Provisional application No. 60/425,145, filed on Nov. 8, 2002.

(51) Int. Cl. C07C 2/02

(52) U.S. Cl. 585/521

(57) ABSTRACT

Active catalyst components, in a process stream from the formation of α-olefins by the catalyzed oligomerization of ethylene using complexes of late transition metals with tridentate ligands as catalyst components, can be accomplished by contacting the process stream with a protic organic compound having a specified pKa.

### FIELD OF THE INVENTION

A process stream from the synthesis of α-olefins from ethylene using a late transition metal complex of a tridentate ligand as (part of) the catalyst system for oligomerizing ethylene is deactivated using a protic organic compound having a specified pKa.

### TECHNICAL BACKGROUND

α-Olefins are important items of commerce, hundreds of millions of kilograms being manufactured yearly. They are useful as monomers for (co)polymerizations and as chemical intermediates for the manufacture of many other materials, for example detergents and surfactants. Presently most α-olefins are made by the catalyzed oligomerization of ethylene by various catalysts, especially certain nickel complexes or aluminum alkyls, see for instance U.S. Pat. No. 4,020,121 and I. Kroschwitz, et al., Ed., Kirk-Othmer Encyclopedia of Chemical Technology, 4^{th} Ed., Vol. 17, John Wiley & Sons, New York, pp. 839-858. Recently, as reported in U.S. Pat. No. 6,103,946, which is hereby incorporated by reference, it has been found that iron complexes of certain tridentate ligands of 2,6-pyridinecarboxaldehyes or 2,6-diacylpyridines are excellent catalysts for the production of α-olefins from ethylene. U.S. patent application Ser. Nos. 2002/0016521 and 2002019575, both of which are hereby included by reference, describe a manufacturing process for α-olefins using these catalysts in which a liquid full continuous stirred tank reactor is used, optionally followed by a final reactor which may be plug flow reactor. No mention is made of deactivation of the process stream with organic compounds.

### SUMMARY OF THE INVENTION

This invention concerns, a process for the preparation of α-olefins by the catalyzed oligomerization of ethylene using as part of a catalyst system a complex late transition metal with a tridentate ligand wherein a process stream comprising said α-olefins and said catalyst system is produced, wherein the improvement comprises, deactivating said catalyst system by adding to said process stream one or more organic compounds (deactivating agents) having a pKa of about 2 to about 20.

### DETAILS OF THE INVENTION

Herein, certain terms are used. Some of them are:

A “hydrocarbyl group” is a univalent group containing only carbon and hydrogen. As examples of hydrocarbyls may be mentioned unsubstituted alkyls, cycloalkyls and aryls. If not otherwise stated, it is preferred that hydrocarbyl groups (and alkyl groups) herein contain 1 to about 30 carbon atoms.

By “substituted hydrocarbyl” herein is meant a hydrocarbyl group that contains one or more substituent groups which are inert under the process conditions to which the compound containing these groups is subjected (e.g., an inert functional group, see below). The substituent groups also do not substantially detrimentally interfere with the oligomerization process or operation of the oligomerization catalyst system. If not otherwise stated, it is preferred that substituted hydrocarbyl groups herein contain 1 to about 30 carbon atoms. Included in the meaning of “substituted” are rings containing one or more heteroatoms, such as nitrogen, oxygen and/or sulfur, and the free valence of the substituted hydrocarbyl may be to the heteroatom. In a substituted hydrocarbyl, all of the hydrogens may be substituted, as in trifluoromethyl.

By “(inert) functional group” herein is meant a group, other than hydrocarbyl or substituted hydrocarbyl, which is inert under the process conditions to which the compound containing the group is subjected. The functional groups also do not substantially deleteriously interfere with any process described herein that the compound in which they are present may take part in. Examples of functional groups include halo (fluoro, chloro, bromo and iodo), and ether such as —OR⁵⁰ wherein R⁵⁰ is hydrocarbyl or substituted hydrocarbyl. In cases in which the functional group may be near a transition metal atom, the functional group alone should not coordinate to the metal atom more strongly than the groups in those compounds that are shown as coordinating to the metal atom, that is they should not displace the desired coordinating group.

By a “cocatalyst” or a “catalyst activator” is meant one or more compounds that react with a transition metal compound to form an activated catalyst species. One such catalyst activator is an “alkyl aluminum compound” which, herein, means a compound in which at least one alkyl group is bound to an aluminum atom. Other groups such as, for example, alkoxide, hydride, an oxygen atom bridging two aluminum atoms, and halogen may also be bound to aluminum atoms in the compound.

By a “linear α-olefin product” is meant a composition predominantly comprising a compound or mixture of compounds of the formula H(CH₂CH₂)_{q}CH═CH₂ wherein q is an integer of 1 to about 18. In most cases, the linear α-olefin product of the present process will be a mixture of compounds having differing values of q of from 1 to 18, with a minor amount of compounds having q values of more than 18. Preferably less than 50 weight percent, and more preferably less than 20 weight percent, of the product will is have q values over 18. The product may further contain small amounts (preferably less than 30 weight percent, more preferably less than 10 weight percent, and especially preferably less than 2 weight percent) of other types of compounds such as alkanes, branched alkenes, dienes and/or internal olefins.

By a “primary carbon group” herein is meant a group of the formula —CH₂—, wherein the free valence—is to any other atom, and the bond represented by the solid line is to a ring atom of a substituted aryl to which the primary carbon group is attached. Thus the free valence—may be bonded to a hydrogen atom, a halogen atom, a carbon atom, an oxygen atom, a sulfur atom, etc. In other words, the free valence—may be to hydrogen, hydrocarbyl, substituted hydrocarbyl or a functional group. Examples of primary carbon groups include —CH₃, —CH₂CH(CH₃)₂, —CH₂Cl, —CH₂C₆H₅, —OCH₃ and —CH₂OCH₃.

By a “secondary carbon group” is meant the group

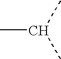

wherein the bond represented by the solid line is to a ring atom of a substituted aryl to which the secondary carbon group is attached, and both free bonds represented by the dashed lines are to an atom or atoms other than hydrogen. These atoms or groups may be the same or different. In other words the free valences represented by the dashed lines may be hydrocarbyl, substituted hydrocarbyl or inert functional groups. Examples of secondary carbon groups include —CH(CH₃)₂, —CHCl₂, —CH(C₆H₅)₂, cyclohexyl, —CH(CH₃)OCH₃, and —CH═CCH₃.

By a “tertiary carbon group” is meant a group of the formula

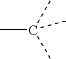

wherein the bond represented by the solid line is to a ring atom of a substituted aryl to which the tertiary carbon group is attached, and the three free bonds represented by the dashed lines are to an atom or atoms other than hydrogen. In other words, the bonds represented by the dashed lines are to hydrocarbyl, substituted hydrocarbyl or inert functional groups. Examples of tetiary carbon groups include —C(CH₃)₃, —C(C₆H₅)₃, —CCl₃, —CF₃, —C(CH₃)₂OCH₃, —C≡CH, —C(CH₃)₂CH═CH₂, aryl and substituted aryl such as phenyl and 1-adamantyl.

By “aryl” is meant a monovalent aromatic group in which the free valence is to the carbon atom of an aromatic ring. An aryl may have one or more aromatic rings, which may be fused, connected by single bonds or other groups.

By “substituted aryl” is meant a monovalent aromatic group substituted as set forth in the above definition of “substituted hydrocarbyl”. Similar to an aryl, a substituted aryl may have one or more aromatic rings which may be fused, connected by single bonds or other groups; however, when the substituted aryl has a heteroaromatic ring, the free valence in the substituted aryl group can be to a heteroatom (such as nitrogen) of the heteroaromatic ring instead of a carbon.

By a “first ring atom in R⁶ and R⁷ bound to an imino nitrogen atom” is meant the ring atom in these groups bound to an imino nitrogen shown in (I), for example

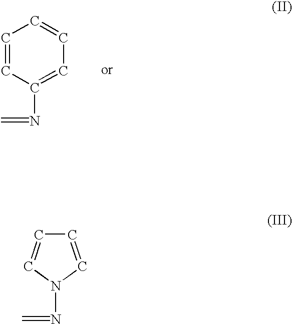

the atoms shown in the 1-position in the rings in (II) and (III) are the first ring atoms bound to an imino carbon atom (other groups which may be substituted on the aryl groups are not shown). Ring atoms adjacent to the first ring atoms are shown, for example, in (IV) and (V), where the open valencies to these adjacent atoms are shown by dashed lines [the 2,6-positions in (IV) and the 2,5-positions in (V)].

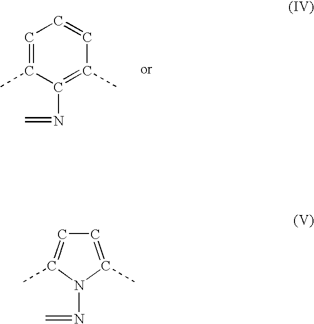

By “pKa” herein is meant the usual meaning, the pH at which a Bronsted acid is half in the protic form and half in the ionized form, in dilute solution. pkas of about 14 or less can be measured by well-known methods in dilute aqueous solution. pkas above about 14 may be measured by methods described in F. G. Bordwell, Acc. Chem. Res., vol. 21, p. 456-463 (1988). Organic compounds within the appropriate pKa range are sometimes called “protic” compounds herein.

By “deactivation” herein is meant that (at least some of) the oligomerization catalyst (system) is no longer able to oligomerize ethylene. In other words the catalyst is rendered inert towards ethylene. The deactivation may be partial so that only some of the oligomerization catalyst is deactivated or all of the oligomerization catalyst is deactivated. Due to the chemical nature of the active catalyst and cocatalyst(s) (if any) present, cocatalyst(s) may also be deactivated by the protic compound used herein. By “complete deactivation” herein is meant all oligomerization catalyst and all cocatalyst(s) are completely deactivated (see below).

By a “tridentate ligand” is meant is neutral organic compound having three heteroatoms (atoms other than carbon and hydrogen) which are in a position to potentially complex with a late transition metal. Such heteroatoms include nitrogen, oxygen, sulfur and phosphorous.

By a “late transition metal” herein is meant a metal of Group 7 through Group 12 of the periodic table (IUPAC notation). Preferred late transition metals are Co and Fe, and Fe is especially preferred.

By an “alkylaluminum compound” herein is meant a compound having at least one alkyl group bound directly to an aluminum atom. Other elements such as halogen (especially chorine) and oxygen may be present in the compound. Useful alkylaluminum compounds include trialkylaluminum compounds such as trimethylaluminum, triethylaluminum and tri-i-butylaluminum, aluminoxanes such as methyl aluminoxanes, and dialkylhaloaluminum compounds such as diethylaluminum chloride and ethylaluminum sesquichloride.

Generally speaking processes to make linear α-olefins with the catalysts described herein are often similar. Ethylene and the metal complex together with optional ingredients solvent and cocatalyst(s) are added and mixed in a vessel. The reaction may then take place in that vessel and possibly other vessels as the process stream moves through the plant. During that time more ethylene and/or metal complex and/or solvent and/or cocatalysts(s) may be added at one or more other points in the process. At some point the synthesis of the α-olefins is complete and/or it is desirable to stop the oligomerization, so the process stream is directed out of the oligomerization reactor(s). Oftentimes at this point excess ethylene is vented or stripped from the process stream and/or the reactive catalyst components are removed by washing with water or other aqueous solution, and then the process stream, which includes linear α-olefins and solvent (if present) is fractionally distilled through a series of distillation columns to isolate pure α-olefins and/or groups of α-olefins. While many variations are possible, most processes have these basic elements.

A preferred tridentate complex herein is an iron or cobalt, especially iron, complex of a 2,6-pyridinedicarboxaldehyebisimine or a 2,6-diacylpyridinebisimine. Such a preferred ligand may have the formula

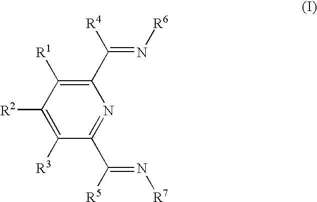

wherein:

R¹, R² and R³ are each independently hydrogen, hydrocarbyl, substituted hydrocarbyl or an inert functional group, provided that any two of R¹, R² and R³ vicinal to one another taken together may form a ring;

R⁴ and R⁵ are each independently hydrogen, hydrocarbyl, substituted hydrocarbyl or an inert functional group;

R⁶ and R⁷ are each independently a substituted aryl having a first ring atom bound to the imino nitrogen, provided that:

in R⁶, a second ring atom adjacent to said first ring atom is bound to a halogen, a primary carbon group, a secondary carbon group or a tertiary carbon group; and further provided that

in R⁶, when said second ring atom is bound to a halogen or a primary carbon group, none, one or two of the other ring atoms in R⁶ and R⁷ adjacent to said first ring atom are bound to a halogen or a primary carbon group, with the remainder of the ring atoms adjacent to said first ring atom being bound to a hydrogen atom; or

in R⁶, when said second ring atom is bound to a secondary carbon group, none, one or two of the other ring atoms in R⁶ and R⁷ adjacent to said first ring atom are bound to a halogen, a primary carbon group or a secondary carbon group, with the remainder of the ring atoms adjacent to said first ring atom being bound to a hydrogen atom; or

in R⁶, when said second ring atom is bound to a tertiary carbon group, none or one of the other ring atoms in R⁶ and R⁷ adjacent to said first ring atom are bound to a tertiary carbon group, with the remainder of the ring atoms adjacent to said first ring atom being bound to a hydrogen atom.

In one preferred compound (I) R⁶ is

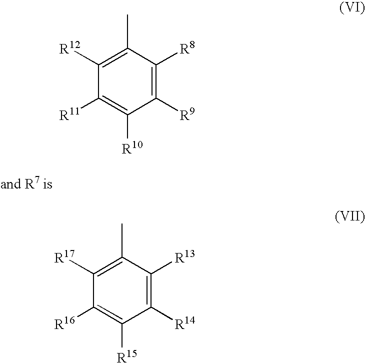

wherein:

R⁸ is a halogen, a primary carbon group, a secondary carbon group or a tertiary carbon group; and

R⁹, R¹⁰, R¹¹, R¹⁴, R¹⁵, R¹⁶ and R¹⁷ are each independently hydrogen, hydrocarbyl, substituted hydrocarbyl or a functional group; provided that:

when R⁸ is a halogen or primary carbon group none, one or two of R¹², R¹³ and R¹⁷ are a halogen or a primary carbon group, with the remainder of R¹², R¹³ and R¹⁷ being hydrogen; or

when R⁸ is a secondary carbon group, none or one of R¹², R¹³ and R¹⁷ is a halogen, a primary carbon group or a secondary carbon group, with the remainder of R¹², R¹³ and R¹⁷ being hydrogen; or

when R⁸ is a tertiary carbon group, none or one of R¹², R¹³ and R¹⁷ is tertiary carbon group, with the remainder of R¹², R¹³ and R¹⁷ being hydrogen; and further provided that any two of R⁸, R⁹, R¹⁰, R¹¹, R¹², R¹³, R¹⁴, R¹⁵, R¹⁶ and R¹⁷ vicinal to one another, taken together may form a ring.

In the above formulas (VI) and (VII), R⁸ corresponds to the second ring atom adjacent to the first ring atom bound to the imino nitrogen, and R¹², R¹³ and R¹⁷ correspond to the other ring atoms adjacent to the first ring atom.

In compounds (I) containing (VI) and (VII), it is particularly preferred that:

if R⁸ is a primary carbon group, R¹³ is a primary carbon group, and R¹² and R¹⁷ are hydrogen; or

if R⁸ is a secondary carbon group, R¹³ is a primary carbon group or a secondary carbon group, more preferably a secondary carbon group, and R¹² and R¹⁷ are hydrogen; or

if R⁸ is a tertiary carbon group (more preferably a trihalo tertiary carbon group such as a trihalomethyl), R¹³ is a tertiary carbon group (more preferably a trihalotertiary group such as a trihalomethyl), and R¹² and R¹⁷ are hydrogen; or

if R⁸ is a halogen, R¹³ is a halogen, and R¹² and R¹⁷ are hydrogen.

In all specific preferred compounds (I) in which (VI) and (VII) appear, it is preferred that R¹, R² and R³ are hydrogen; and/or R⁴ and R⁵ are methyl. It is further preferred that:

R⁹, R¹⁰, R¹¹, R¹², R¹⁴, R¹⁵, R¹⁶ and R¹⁷ are all hydrogen; R¹³ is methyl; and R⁸ is a primary carbon group, more preferably methyl; or

R⁹, R¹⁰, R¹¹, R¹², R¹⁴, R¹⁵, R¹⁶ and R¹⁷ are all hydrogen; R¹³ is ethyl; and R⁸ is a primary carbon group, more preferably ethyl; or

R⁹, R¹⁰, R¹¹, R¹², R¹⁴, R¹⁵, R¹⁶ and R¹⁷ are all hydrogen; R¹³ is isopropyl; and R⁸ is a primary carbon group, more preferably isopropyl; or

R⁹, R¹⁰, R¹¹, R¹², R¹⁴, R¹⁵, R¹⁶ and R¹⁷ are all hydrogen; R¹³ is n-propyl; and R⁸ is a primary carbon group, more preferably n-propyl; or

R⁹, R¹⁰, R¹¹, R¹², R¹⁴, R¹⁵, R¹⁶ and R¹⁷ are all hydrogen; R¹³ is chloro; and R⁸ is a halogen, more preferably chloro; or

R⁹, R¹⁰, R¹¹, R¹², R¹⁴, R¹⁵, R¹⁶ and R¹⁷ are all hydrogen; R¹³ is trihalomethyl, more preferably trifluoromethyl; and R⁸ is a trihalomethyl, more preferably trifluoromethyl.

In another-preferred embodiment of (I), R⁶ and R⁷ are, respectively

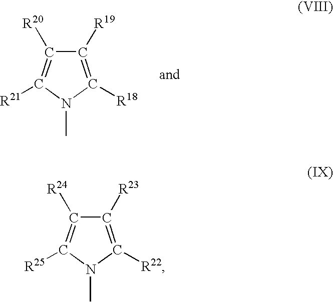

wherein:

R¹⁸ is a halogen, a primary carbon group, a secondary carbon group or a tertiary carbon group; and

R¹⁹, R²⁰, R²³ and R²⁴ are each independently hydrogen, hydrocarbyl, substituted hydrocarbyl or a functional group; and

provided that:

when R¹⁸ is a halogen or primary carbon group none, one or two of R²¹, R²² and R²⁵ are a halogen or a primary carbon group, with the remainder of R²¹, R²² and R²⁵ being hydrogen; or

when R¹⁸ is a secondary carbon group, none or one of R²¹, R²² and R²⁵ is a halogen, a primary carbon group or a secondary carbon group, with the remainder of R²¹, R²² and R²⁵ being hydrogen;

when R¹⁸ is a tertiary carbon group, none or one of R²¹, R²² and R²⁵ is a tertiary carbon group, with the remainder of R²¹, R²² and R²⁵ being hydrogen;

and further provided that any two of R¹⁸, R¹⁹, R²⁰, R²¹, R²², R²³, R²⁴ and R²⁵ vicinal to one another, taken together may form a ring.

In the above formulas (VIII) and (IX), R¹⁸ corresponds to the second ring atom adjacent to the first ring atom bound to the imino nitrogen, and R²¹, R²² and R²⁵ correspond to the other ring atoms adjacent to the first ring atom.

In compounds (I) containing (VIII) and (IX), it is particularly preferred that:

if R¹⁸ is a primary carbon group, R²² is a primary carbon group, and R²¹ and R²⁵ are hydrogen; or

if R¹⁸ is a secondary carbon group, R²² is a primary carbon group or a secondary carbon group, more preferably a secondary carbon group, and R²¹ and R²⁵ are hydrogen; or

if R¹⁸ is a tertiary carbon group (more preferably a trihalo tertiary carbon group such as a trihalomethyl), R²² is a tertiary carbon group (more preferably a trihalotertiary group such as a trihalomethyl), and R²¹ and R²⁵ are hydrogen; or

if R¹⁸ is a halogen, R²² is a halogen, and R²¹ and R²⁵ are hydrogen.

In all specific preferred compounds (I) in which (VII) and (IX) appear, it is preferred that R¹, R² and R³ are hydrogen; and/or R⁴ and R⁵ are methyl. It is further preferred that:

R¹⁹, R²⁰, R²¹, R²³ and R²⁴ are all hydrogen; R²² is methyl; and R¹⁸ is a primary carbon group, more preferably methyl; or

R¹⁹, R²⁰ , R²¹ , R²³ and R²⁴ are all hydrogen; R²² is ethyl; and R¹⁸ is a primary carbon group, more preferably ethyl; or

R¹⁹, R²⁰, R²¹, R²³ and R²⁴ are all hydrogen; R²² is isopropyl; and R¹⁸ is a primary carbon group, more preferably isopropyl; or

R¹⁹, R²⁰, R²¹, R²³ and R²⁴ are all hydrogen; R²² is n-propyl; and R¹⁸ is a primary carbon group, more preferably n-propyl; or

R¹⁹, R²⁰, R²¹, R²³ and R²⁴ are all hydrogen; R²² is chloro or bromo; and R¹⁸ is a halogen, more preferably chloro or bromo.

Compound (I) and its iron complexes (the oligomerization catalyst) may be prepared by a variety of methods, see for instance previously incorporated U.S. Pat. No. 5,955,555 and WO99/02472, as well as WO99/50273 (equivalent to U.S. patent application Ser. No. 09/277,910, filed Mar. 29, 1999) and WO00/08034, all of which are also included by reference.

The use of 2,6-pyridinecarboxaldehyde or 2,6-diacylpyridine complexes as ethylene oligomerization and/or polymerization catalysts, and the general conditions for such reactions, including temperature, pressure, supportation of the iron complex (if desired), useful cocatalysts and amounts, much of which is useful herein, may be found in U.S. Pat. Nos. 5,955,555, 6,103,946, World Patent Applications 02/06192, 02/12151, 01/58874 and 02/00339, and U.S. Provisional Patent Applications 60/285,554 filed Apr. 20, 2001 (CL1844 PRV1) and 60/411,449 filed Sep. 17, 2003 (CL2151 PRV), all of which are hereby included by reference. Another type of useful tridentate late transition metal complex is found in World Patent Application 02/34710 which is also hereby included by reference.

The process may be run in an inert solvent such as a hydrocarbon. Useful hydrocarbons include alkanes such as heptane, or nonane, or aromatic hydrocarbons such as toluene or xylene. Preferably the solvent has a boiling point that allows it be readily separated by distillation from the α-olefins produced in the process. The “solvent” for the process may be some or all of the α-olefins produced in the process. They may be formed in situ and/or added at some point during the process.

Cocatalysts are also often used in the oligomerization process. Typically these cocatalysts are compounds that are alkylating or hydriding agents such as one or more alkylaluminum compounds or metal hydrides, respectively. Alkylaluminum compounds are probably the most common type of cocatalysts. These cocatalysts are believed to react with the late transition metal complex to form complexes, which are the actual active oligomerization catalysts. These active oligomerization catalysts are believed to (mostly) have alkyl and/or hydride groups bonded to the transition metal atom. Typically these cocatalysts are added in molar excess (of the transition metal complex) to both ensure reaction with the late transition metal complex and to remove the last traces of catalyst poisons from the reaction system. In order to completely deactivate at the catalyst, it is preferred to add a stoichiometric excess of the organic protic deactivating agent, that is more than one mole of “active protons” per equivalent of activating groups (alkyl, hydride, etc.). For example if the deactivating compound was an alcohol, R⁶⁰OH, wherein R⁶⁰ is alkyl, it would take 3 moles of R⁶⁰OH to completely deactivate one mole of alkylaluminum compound R⁶¹₃Al wherein R⁶¹ is alkyl. Therefore a stoichiometric amount, preferably a stoichiometric excess, of deactivating agent is used to completely deactivate the process stream.

The amount of deactivating agent needed for partial or complete deactivation of the catalyst components may be easily determined by titration of the appropriate process stream with the deactivating agent itself.

The deactivating agent has a pKa of about 2 to about 20, preferably about 3 to about 18. Useful types of deactivating agents include alcohols, phenols (compounds having hydroxy groups bound to aromatic ring carbon atoms), carboxylic acids, and relatively acidic aldehydes. Alcohols and carboxylic acids are preferred deactivating agents. The deactivating agent may be monofunctional (have one protic group present) or polyfunctional (such as a diol or triol). Monofunctional deactivating agents are preferred. Preferably the deactivating agent and its reaction product(s) with any of the compounds it may deactivate [for example deactivating R⁶¹₃Al with R⁶⁰OH may give formation of (R⁶⁰O)₃Al and R⁶¹H; R⁶¹H is typically a lower alkane and therefore volatile and inert] are soluble in the process stream. The process stream will usually predominantly be solvent (if used) and product α-olefins.

Typically the series of α-olefins produced in this type of process is separated (into pure olefins or groups of olefins) by fractional distillation through multiple distillation columns. Preferably the deactivating agent has a boiling point of about 170° C. or more at a pressure of 2 kPa, more preferably about 220° C. or more at a pressure of 2 kPa, and especially preferably about 250° C. or more at a pressure of 2 kPa. When such a low volatility compound is used as the deactivating compound it typically will not codistill with any of the usually isolated olefin fractions, thereby eliminating the problem of having an undesirable impurity in one or more of the purified product streams. It also will not appreciably contaminate any gaseous recycle streams, such as a recycle ethylene stream, thereby reducing or even eliminating the purification capacity needed to purify such streams. Finally since the residues from the catalyst deactivation typically will end up in the still bottoms from the final distillation column, and these are often burned for their fuel value, there is no separate waste stream of catalyst deactivation products as there is, for example, using an aqueous based wash.

The deactivating agent may be added to the process stream at any point after it is desired to stop the oligomerization reaction. Typically this will be on exiting the (final) reactor in which oligomerization takes place and before entering the first product distillation column. It may be added before or after excess ethylene in the process stream is removed (flashed off), or may be added after some of the excess ethylene is flashed off. Since the deactivating compound may have a low volatility, in that instance it will not appreciably contaminate the recycle ethylene. The deactivating agent, particularly if it is a liquid, may be added neat to the process stream, or it may be added as a solution in a solvent, particularly if the solvent is a compounds or compounds already present in the process (solvent in the process or one or more α-olefins). Preferably the deactivating agent is mixed with the process stream so that, particularly if complete deactivation is desired, the agent will contact all “parts” of that stream.

Useful deactivating agents include octadecanol, stearic acid, bisphenol-A, saccharin, sulfanilic acid, thioacetic acid, ethylene glycol, 1-napthoic acid, and 1-octacosanol. Since many high boiling “compounds” are sold as mixtures because they are difficult to purify by distillation or other means, such mixtures are also useful, and sometimes preferred for economic reasons (lower cost).

What is claimed is:

1. A process for the preparation of α-olefins by the catalyzed oligomerization of ethylene using as part of a catalyst system a complex of a late transition metal with a tridentate ligand wherein a process stream comprising said α-olefins and said catalyst system is produced, wherein the improvement comprises, deactivating said catalyst system by adding to said process stream one or more organic compounds having a pKa of about 2 to about 20.

2. The process as recited in claim 1 wherein said late transition metal is iron and said ligand is a 2,6-pyridinedicarboxaldehyebisimine or a 2,6-diacylpyridinebisimine.

3. The process as recited in claim 2 wherein said ligand has the formula 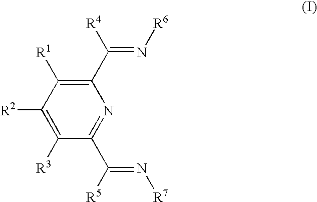
wherein:
R¹, R² and R³ are each independently hydrogen, hydrocarbyl, substituted hydrocarbyl or an inert functional group, provided that any two of R¹, R² and R³ vicinal to one another taken together may form a ring;
R⁴ and R⁵ are each independently hydrogen, hydrocarbyl, substituted hydrocarbyl or an inert functional group;
R⁶ and R⁷ are each independently a substituted aryl having a first ring atom bound to the imino nitrogen, provided that:
in R⁶, a second ring atom adjacent to said first ring atom is bound to a halogen, a primary carbon group, a secondary carbon group or a tertiary carbon group; and further provided that
in R⁶, when said second ring atom is bound to a halogen or a primary carbon group, none, one or two of the other ring atoms in R⁶ and R⁷ adjacent to said first ring atom are bound to a halogen or a primary carbon group, with the remainder of the ring atoms adjacent to said first ring atom being bound to a hydrogen atom; or
in R⁶, when said second ring atom is bound to a secondary carbon group, none, one or two of the other ring atoms in R⁶ and R⁷ adjacent to said first ring atom are bound to a halogen, a primary carbon group or a secondary carbon group, with the remainder of the ring atoms adjacent to said first ring atom being bound to a hydrogen atom; or
in R⁶, when said second ring atom is bound to a tertiary carbon group, none or one of the other ring atoms in R⁶ and R⁷ adjacent to said first ring atom are bound to a tertiary carbon group, with the remainder of the ring atoms adjacent to said first ring atom being bound to a hydrogen atom.

4. The process as recited in claim 3 wherein R⁶ is 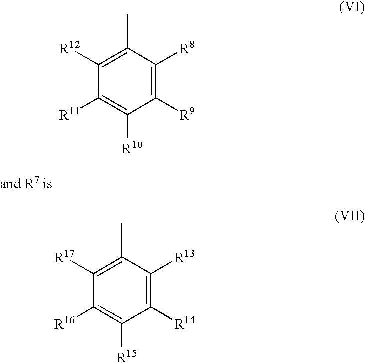
wherein:
R⁸ is a halogen, a primary carbon group, a secondary carbon group or a tertiary carbon group; and
R⁹, R¹⁰, R¹¹, R¹⁴, R¹⁵, R¹⁶ and R¹⁷ are each independently hydrogen, hydrocarbyl, substituted hydrocarbyl or a functional group; provided that:
when R⁸ is a halogen or primary carbon group none, one or two of R¹², R¹³ and R¹⁷ are a halogen or a primary carbon group, with the remainder of R¹², R¹³ and R¹⁷ being hydrogen; or
when R⁸ is a secondary carbon group, none or one of R¹², R¹³ and R¹⁷ is a halogen, a primary carbon group or a secondary carbon group, with the remainder of R¹², R¹³ and R¹⁷ being hydrogen; or
when R⁸ is a tertiary carbon group, none or one of R¹², R¹³ and R¹⁷ is tertiary carbon group, with the remainder of R¹², R¹³ and R¹⁷ being hydrogen; and further provided that any two of R⁸, R⁹, R¹⁰, R¹¹, R¹², R¹³, R¹⁴, R¹⁵, R¹⁶ and R¹⁷ vicinal to one another, taken together may form a ring.

5. The process as recited in claim 5 wherein:
R¹, R² and R³ are hydrogen;
R⁴ and R⁵ are methyl;
R⁹, R¹⁰, R¹¹, R¹², R¹⁴, R¹⁵, R¹⁶ and R¹⁷ are all hydrogen, R¹³ is methyl, and R⁸ is methyl; or
R⁹, R¹⁰, R¹¹, R¹², R¹⁴, R¹⁵, R¹⁶ and R¹⁷ are all hydrogen, R¹³ is ethyl, and R⁸ is ethyl; or
R⁹, R¹⁰, R¹¹, R¹², R¹⁴, R¹⁵, R¹⁶ and R¹⁷ are all hydrogen, R¹³ is isopropyl, and R⁸ is isopropyl; or
R⁹, R¹⁰, R¹¹, R¹², R¹⁴, R¹⁵, R¹⁶ and R¹⁷ are all hydrogen, R¹³ is n-propyl, and R⁸ is n-propyl; or
R⁹, R¹⁰, R¹¹, R¹², R¹⁴, R¹⁵, R¹⁶ and R¹⁷ are all hydrogen, R¹³ is chloro, and R⁸ is chloro; or
R⁹, R¹⁰, R¹¹, R¹², R¹⁴, R¹⁵, R¹⁶ and R¹⁷ are all hydrogen, R¹³ is trifluoromethyl, and R⁸ is trifluoromethyl.

6. The process as recited in claim 1 wherein one or more alkylating or hydriding agents are present.

7. The process as recited in claim 1 wherein one or more alkylaluminum compounds are also present.

8. The process as recited in claim 1 wherein said pKa is about 3 to about 18.

9. The process as recited in claim 1 wherein said organic compound is an alcohol, phenol, or carboxylic acid.

10. The process as recited in claim 3 wherein said organic compound is an alcohol, phenol, or carboxylic acid.

11. The process as recited in claim 7 wherein said organic compound is an alcohol, phenol, or carboxylic acid.

12. The process as recited in claim 9 wherein said organic compound is an alcohol.

13. The process as recited in claim 9 wherein said organic compound is a carboxylic acid.

14. The process as recited in claim 1 wherein said organic compound is monofunctional.

15. The process as recited in claim 1 wherein a stoichiometric excess of said organic compound is added.
